(19) United States
(12) Patent Application Publication
(10) Pub. No.: US 2008/0319039 A1
(43) Pub. Date: Dec. 25, 2008

(54) UNIT DOSAGE FORMS OF TEMOZOLOMIDE

(76) Inventors: Jacqueline Rose Bersch, Metuchen, NJ (US); Mark Manzo, Bethlehem, PA (US); Sumant Ramachandra, East Brunswick, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/767,847

(22) Filed: Jun. 25, 2007

(51) Int. Cl. A61K 31/4188; A61P 35/00

(52) U.S. Cl. 514/393

(57) ABSTRACT

This invention relates to unit dosage forms of temozolomide. These unit dosage forms are particularly well-suited for decreasing the pill burden and increasing patient compliance. The invention also relates to methods of treating proliferative disorders in a patient with these unit dosage forms. The invention additionally relates to kits comprising these unit dosage forms.

### CROSS REFERENCE TO RELATED APPLICATIONS

This application claims priority benefits of provisional application 60/816,623 filed Jun. 26, 2006, the entire contents of which are incorporated herein in their entirety.

### FIELD OF THE INVENTION

This invention relates to unit dosage forms of temozolomide. These unit dosage forms are particularly well-suited for decreasing the pill burden and increasing patient compliance. The invention also relates to methods of treating proliferative disorders in a patient with these unit dosage forms. The invention additionally relates to kits comprising these unit dosage forms.

### BACKGROUND OF THE INVENTION

Brain tumors comprise approximately 2% of all malignant diseases. Stupp et al., J. Clin. One., 20(5):1375-1382 (2002). More than 17,000 cases are diagnosed every year in the United States, with approximately 13,000 associated deaths. The standard protocol for treating a malignant glioma involves cytoreduction through surgical resection, when feasible, followed by radiotherapy (RT) with or without adjuvant chemotherapy. Stupp et al., supra.

A chemotherapeutic agent approved for treating brain tumors is temozolomide (Schering Corp. under the trade name of Temodar® in the United States and Temodal® in Europe). The chemical name for temozolomide is 3,4-dihydro-3-methyl-4-oxoimidazo[5,1-d]-as-tetrazine-8-carboxamide (see U.S. Pat. No. 5,260,291). The cytotoxicity of temozolomide or its metabolite, MTIC (3-methyl-(triazen-1-yl)imidazole-4-carboxamide), is thought to be primarily due to alkylation of DNA. Alkylation (methylation) occurs mainly at the O⁶ and N⁷ positions of guanine.

Temodar® capsules are currently indicated in the United States for the treatment of adult patients with newly diagnosed gliobastoma multiforme as well as refractory anaplastic astrocytoma, i.e. patients at first relapse who have experienced disease progression on a drug regimen containing a nitrosourea and procarbazine. Temodal® is currently approved in Europe for the treatment of patients with malignant glioma, such as glioblastoma multiforme or anaplastic astrocytoma for newly diagnosed patients as well as those showing recurrence or progression after standard therapy.

A typical regimen for patients with glioma taking temolozomide consists of two phases, a concomitant phase, followed by a maintenance phase. In the concomitant phase, the patient receives an oral administration of temozolomide at 75 mg/m² (approximately 140 mg for a patient having a Body Surface Area (BSA) between 1.8 and 1.9 m²) for 42 days concomitant with RT. See Table 1. Four weeks after completing the concomitant phase, the patient receives 6 cycles of maintenance treatment. In the first maintenance cycle, temozolomide is administered at 150 mg/m² (approximately 280 mg for a patient having a BSA between 1.8 and 1.9 m²) once daily for five days followed by 23 days without treatment. The dosage may be escalated to 200 mg/m² (approximately 360 mg for a patient having a BSA between 1.8 and 1.9 ml) for the first 5 days of each subsequent cycle.

Currently, the capsule formulations of temozolomide contain 5, 20, 100 or 250 mg of temozolomide. Given a therapeutic dose of 150 mg/mg, a patient having a BSA of between 1.8 and 1.9 would need to consume 4 capsules per day to receive the approximately 280 mg of temozolomide dosage (1×250 mg, 1×20 mg and 2×5 mg). See Table 2. Such a high pill burden would likely result in poor patient compliance with therapies that require self-administration of temozolomide, thus producing sub-optimal therapeutic benefit of the drug. Other factors that may affect the patient's compliance with self-administering the appropriate number of pills is the intensity of the treatment regimens for gliomas, including brain surgery and RT. Furthermore, the number of different medications that the patient may have to take to alleviate the side effects of the surgery or RT or to remedy or alleviate other unrelated conditions further exacerbate the already high pill burden. In addition, the patient is at risk for cognitive deficits and compromised neurological status as a result of the intense treatment regimens.

Thus, there is a need to improve the unit dosage form of temozolomide. Such an improved dosage form would reduce the pill burden and increase patient compliance.

TABLE 1
Daily Dose Calculations by Body Surface Area (BSA)

| Total BSA | 75 mg/m² | 100 mg/m² | 150 mg/m² | 200 mg/m² |
|---|---|---|---|---|
| (m²) | (mg daily) | (mg daily)* | (mg daily) | (mg daily) |
| 1.0 | 75 | 100 | 150 | 200 |
| 1.1 | 82.5 | 110 | 165 | 220 |
| 1.2 | 90 | 120 | 180 | 240 |
| 1.3 | 97.5 | 130 | 195 | 260 |
| 1.4 | 105 | 140 | 210 | 280 |
| 1.5 | 112.5 | 150 | 225 | 300 |
| 1.6 | 120 | 160 | 240 | 320 |
| 1.7 | 127.5 | 170 | 255 | 340 |
| 1.8 | 135 | 180 | 270 | 360 |
| 1.9 | 142.5 | 190 | 285 | 380 |
| 2.0 | 150 | 200 | 300 | 400 |
| 2.1 | 157.5 | 210 | 315 | 420 |
| 2.2 | 165 | 220 | 330 | 440 |
| 2.3 | 172.5 | 230 | 345 | 460 |
| 2.4 | 180 | 240 | 360 | 480 |
| 2.5 | 187.5 | 250 | 375 | 500 |

*100 mg/m² daily represents the current minimum dosage for patients on maintenance dosing.

TABLE 2
Suggested Currently Available Capsule Combinations
Based on Daily Dose in Adults

| | |
|---|---|
| | Number of Daily Capsules |
| Total Daily Dose | by Strength |

| (mg) | 250 | 100 | 20 | 5 |
|---|---|---|---|---|
| 75 | 0 | 0 | 3 | 3 |
| 82.5 | 0 | 0 | 4 | 0 |
| 90 | 0 | 0 | 4 | 2 |
| 97.5 | 0 | 1 | 0 | 0 |
| 105 | 0 | 1 | 0 | 1 |
| 112.5 | 0 | 1 | 0 | 2 |
| 120 | 0 | 1 | 1 | 0 |
| 127.5 | 0 | 1 | 1 | 1 |
| 135 | 0 | 1 | 1 | 3 |
| 142.5 | 0 | 1 | 2 | 0 |
| 150 | 0 | 1 | 2 | 2 |
| 157.5 | 0 | 1 | 3 | 0 |
| 165 | 0 | 1 | 3 | 1 |
| 172.5 | 0 | 1 | 3 | 2 |
| 180 | 0 | 1 | 4 | 0 |
| 187.5 | 0 | 1 | 4 | 1 |
| 195 | 0 | 1 | 4 | 3 |
| 200 | 0 | 2 | 0 | 0 |
| 210 | 0 | 2 | 0 | 2 |
| 220 | 0 | 2 | 1 | 0 |
| 225 | 0 | 2 | 1 | 1 |
| 240 | 0 | 2 | 2 | 0 |
| 255 | 1 | 0 | 0 | 1 |
| 260 | 1 | 0 | 0 | 2 |
| 270 | 1 | 0 | 1 | 0 |
| 280 | 1 | 0 | 1 | 2 |
| 285 | 1 | 0 | 1 | 3 |
| 300 | 0 | 3 | 0 | 0 |
| 315 | 0 | 3 | 0 | 3 |
| 320 | 0 | 3 | 1 | 0 |
| 330 | 1 | 0 | 4 | 0 |
| 340 | 0 | 3 | 2 | 0 |
| 345 | 0 | 3 | 2 | 1 |
| 360 | 0 | 3 | 3 | 0 |
| 370 | 1 | 1 | 1 | 0 |
| 380 | 1 | 1 | 1 | 2 |
| 400 | 0 | 4 | 0 | 0 |

### SUMMARY OF THE INVENTION

The present invention provides an improved unit dosage form of temozolomide comprising about 140 mg temozolomide. The present invention also provides an improved unit dosage form of temozolomide comprising about 180 mg of temozolomide. These unit dosage forms reduce the pill burden and increase patient compliance.

In some embodiments, the unit dosage forms are in the form of a capsule. In some embodiments, the capsules are color-coded. In some embodiments, the 140 mg temozolomide capsule differs in color from the 180 mg temozolomide capsule.

The present invention also provides methods of increasing patient compliance with a regimen by administering these unit dosage forms.

The present invention also provides methods of treating proliferative disorders with these unit dosage forms. In some embodiments, the proliferative disorder is selected from a glioma, melanoma, a lung cancer, a lymphoma, a head and neck cancer, ovarian cancer, colorectal and/or colon cancer or esophageal cancer, or other solid tumor or hematologic malignancy.

In some embodiments, the methods of treating proliferative disorders comprise administering the unit dosage forms according to a dosing regimen. In some embodiments, the regimen is based upon the methylation state of the promoter region of the O⁶-methylguanine-DNA transferase (MGMT) gene in a sample obtained from the patient. In other embodiments, the regimen is based upon the presence or absence of the MGMT protein in a sample obtained from the patient. In other embodiments, the regimen is based upon the level or enzymatic activity of the MGMT protein detected in a sample obtained from the patient. In other embodiments, the regimen is based upon the presence (or level) of messenger RNA of MGMT in a sample obtained from the patient.

The present invention also provides kits comprising these unit dosage forms. In some embodiments, the kits comprise both unit dosage forms. In some embodiments, the kits further comprise one or more unit dosage forms comprising about 5, 20 or 100 mg of temozolomide. In some embodiments, the kit is a blister pack.

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

In order to further define the invention, the following terms and definitions are provided herein.

The term “pharmaceutically acceptable carrier or adjuvant” refers to a non-toxic carrier or adjuvant that may be administered to a patient, together with temolozimide, and that does not destroy the pharmacological activity thereof.

The term “color-coded capsule” refers to a capsule in which any portion or all of the capsule is colored and the color represents a particular meaning, e.g. a given capsule color represents a specific dosage amount of temozolomide.

The term “treating” or “treatment” is intended to mean mitigating or alleviating the symptoms a cell proliferative disorder in a mammal such as a human or the improvement of an ascertainable measurement associated with a cell proliferative disorder.

The term “patient” refers to an animal including a mammal (e.g., a human).

The term “proliferative disorder” may be a neoplasm. Such neoplasms are either benign or malignant.

The term “neoplasm” refers to a new, abnormal growth of cells or a growth of abnormal cells that reproduce faster than normal. A neoplasm creates an unstructured mass (a tumor) which can be either benign or malignant.

The term “benign” refers to a tumor that is noncancerous, e.g., its cells do not invade surrounding tissues or metastasize to distant sites.

The term “malignant” refers to a tumor that is cancerous, metastastic, invades contiguous tissue or is no longer under normal cellular growth control.

The term “brain tumor” includes glioma, glioblastoma multiforme, ependymoma; astrocytoma, medulloblastoma, neuroglioma, oligodendroglioma and meningioma.

The term “sample” refers to a specimen that can obtained as or isolated from normal or tumor tissue, brain tissue, cerebrospinal fluid, blood, plasma, serum, urine, stool, saliva lymph, lymph nodes, spleen, liver, bone marrow, or any other biological specimen containing either MGMT protein or nucleic acid of the MGMT gene.

The term “MGMT” refers to O⁶-methylguanine-DNA methyltransferase. MGMT is also known as an O⁶-alkylguanine-DNA-alkyltransferase (AGAT).

The term “GM-CSF” means a protein which (a) has an amino acid sequence that is substantially identical to the sequence of mature (i.e., lacking a signal peptide) human GM-CSF described by Lee et al., Proc. Natl. Acad. Sci. U.S.A., 82:4360 (1985) and (b) has biological activity that is common to native GM-CSF.

The term “substantial identity of amino acid sequences” means that the sequences are identical or differ by one or more amino acid alterations (deletions, additions, substitutions) that do not substantially impair biological activity.

The term “kit” refers to one or more containers for containing the unit dosage forms of the present invention.

### Unit Dosage Forms

The present invention provides a unit dosage form of temozolomide comprising about 140 mg temozolomide and a pharmaceutically acceptable carrier. The present invention also provides a unit dosage form of temozolomide comprising about 180 mg of temozolomide and a pharmaceutically acceptable carrier. In some embodiments, the unit dosage form is suitable for oral administration.

Pharmaceutically acceptable carriers that may be used in the dosage forms of the present invention include those well known in the art (see, e.g., Remington's Pharmaceutical Sciences, 16th Ed., Mac Publishing Company (1980)). Such pharmaceutically acceptable carriers may include other medicinal agents, carriers, genetic carriers, adjuvants, excipients, inert diluents, lubricating agents, etc., such as human serum albumin or plasma preparations. Non-aqueous vehicles such as fixed oils and ethyl oleate are also useful herein. Examples of diluents include calcium carbonate, potato starch, alginic acid, and lactose. Examples of inert diluents include lubricating agents, such as magnesium stearate. Preferably, the pharmaceutically acceptable carriers are lactate anhydrous, colloidal silicon dioxide, sodium starch glycolate, tartartic acid and stearic acid.

The unit dosage forms according to the present invention may be prepared in the form of tablets, pills, granules, dispersible powders or capsules. Preferably, the unit dosage forms are in the form of capsules. In some embodiments, the capsules are color-coded for easy identification. In some embodiments, the 140 mg temozolomide capsule differs in color from the 180 mg temozolomide capsule. In some embodiments, each capsule strength has a different color, thus indicating dosage of temozolomide.

### Methods of Increasing Patient Compliance

The unit dosage forms of the present invention may be used to increase patient compliance by reducing the pill burden. In some embodiments, the present invention provides methods of increasing patient compliance by administering to the patient one or more unit dosage forms containing about 140 mg or 180 mg of temozolomide. In some embodiments, the methods further comprise administering one or more unit dosage forms containing about 5 mg, 20 mg or 100 mg of temozolomide.

The unit dosage forms may be administered to a patient according to a dosing regimen and/or dosing schedule. Non-limiting examples of dosing regimens and schedules are illustrated in Table 3.

TABLE 3
TMZ Dosing Regimens and Dose Intensity

| Regimen | | | Total Dose | Dose/wk | Dose |
|---|---|---|---|---|---|
| No. | Dosing Regimen | Dosing Schedule | (mg/m²/4 wks) | (mg/m²) | Intensity |
| 1 | 5/28 | 150-200 mg/m², 5 days/28 day | 1000 | 250 | 1 |
| | | cycle (200 mg) | | | |
| 2 | High doses 250 mg/m² | 250 mg/m², 5/28, concomitant | 1250 | 312 | 12 |
| | for 5/28 | w/a growth factor | | | |
| 3 | 14/28 | 100 mg/m², 14 days/28 day | 1400 | 350 | 1.4 |
| | | cycle | | | |
| 4 | High doses 300 mg/m² | 300 mg/m², 5/28, concomitant | 1500 | 375 | 1.5 |
| | for 5/28 | w/a growth factor | | | |
| 5 | 21/28 | 75 mg/m², 21 days/28 day | 1575 | 393.75 | 1.6 |
| | | cycle | | | |
| 6 | 42/56 | 75 mg/m², 6 wks/8 wk cycle | 3150 | 393.75 | 1.6 |
| 7 | 21/28 | 85 mg/m², 21 days/28 day | 1785 | 446.25 | 1.8 |
| | | cycle | | | |
| 8 | High doses 350 mg/m² | 350 mg/m², 5/28, concomitant | 1750 | 437.5 | 1.8 |
| | for 5/28 | w/a growth factor | | | |
| 9 | 14 on/7 off | 100 mg/m², 14 days/21 day | 1400* | 467 | 1.9 |
| | | cycle | | | |
| 10 | High doses 400 mg/m² | 400 mg/m², 5/28, concomitant | 2000 | 500 | 2.0 |
| | for 5/28 | w/a growth factor | | | |
| 11 | 7/7 | 150 mg/m², 7 days/14 day | 2100 | 525 | 2.1 |
| | | cycle | | | |
| 12 | 21/28 | 100 mg/m², 21 days/28 day | 2100 | 525 | 2.1 |
| | | cycle | | | |
| 13 | 14/28 | 150 mg/m², 14 days/28 day | 2100 | 525 | 2.1 |
| | | cycle | | | |
| 14 | Continuous | 75 mg/m², daily | 2100 | 525 | 2.1 |
| | dosing | | | | |
| 15 | High doses 450 mg/m² | 450 mg/m², 5/28 concomitant | 2250 | 562.5 | 2.25 |
| | for 5/28 | w/a growth factor | | | |
| 16 | 14 on/7 off | 150 mg/m², 14 days/21 day | 2100* | 700 | 2.8 |
| | | cycle | | | |
| 17 | Continuous | 100 mg/m², daily | 2800 | 700 | 2.8 |
| | dosing | | | | |
| 18 | High doses 250 mg/m² | 250 mg/m², 7/7, concomitant | 3500 | 875 | 3.5 |
| | for 7/7 | w/a growth factor | | | |
| 19 | High doses 300 mg/m² | 300 mg/m², 7/7, concomitant | 4200 | 1050 | 4.2 |
| | for 7/7 | w/a growth factor | | | |

*Represents total dose received in 3 week cycle

The dosing regimens of Table 3 may be simplified by using the unit dosage forms of the present invention. Suggested capsule combinations using the unit dosage forms of the present invention are illustrated in Table 4. In 18 of 29 daily doses listed in Table 4, the suggested capsule combinations of the present invention are lower than the currently available capsule combinations. See Table 5. Importantly, the suggested capsule combinations of the present invention increase in only 2 daily doses (see, 255 mg and 370 mg in Table 2 compared to Table 4). By simplifying the dosing regimens, the pill burden on the patient is significantly reduced.

In particular, a patient on a 195 mg of temozolomide per day regimen would need to consume 8 of the current capsule formulations to achieve the therapeutic dosage (i.e., 1×100 mg, 4×20 mg and 3×5 mg). See Tables 2 and 5. However, using the unit dosage forms of the present invention, that patient would need to consume only 4 units dosage forms (i.e., 1×180 mg and 3×5 mg)—See Tables 4 and 5. As another example, a patient on a 280 mg of temozolomide per day regimen would need to consume 4 of the current capsule formulations to achieve the therapeutic dosage (i.e., 1×250 mg, 1×20 mg and 2×5 mg). See Tables 2 and 5. However, using the unit dosage forms of the present invention, that patient would need to consume only 2 unit dosage forms (i.e., 2×140 mg). See Tables 4 and 5. As yet another example, a patient on a 380 mg of temozolomide per day regimen would need to consume 5 of the current capsule formulations to achieve the therapeutic dosage (i.e., 1×250 mg, 1×100 mg, 1×20 mg and 2×5 mg). See Tables 2 and 5. In contrast, that patient would need to consume only 3 unit dosage forms of the present invention (i.e., 2×180 mg and 1×20 mg). See Tables 4 and 5.

Table 6 further illustrates how the unit dosage forms of the present invention decrease the pill burden on the patient. As shown in Table 6, the number of capsules is lower in 28 out of 45 regimens when the patient receives the unit dosage forms of the present invention. See Table 6. Importantly, the number increases in only 8 of the remaining 17 regimens.

TABLE 4
Suggested Capsule Combinations Based on Daily Dose in Adults

| | Number of Daily | | | | |
|---|---|---|---|---|---|
| Total Daily Dose | Capsules by Strength | | | | |
| (mg)* | 180 | 140 | 100 | 20 | 5 |
| 135 | 0 | 1 | 0 | 0 | 0 |
| 142.5 | 0 | 1 | 0 | 0 | 0 |
| 150 | 0 | 1 | 0 | 0 | 2 |
| 157.5 | 0 | 1 | 0 | 1 | 0 |
| 165 | 0 | 1 | 0 | 1 | 1 |
| 172.5 | 0 | 1 | 0 | 1 | 2 |
| 180 | 1 | 0 | 0 | 0 | 0 |
| 187.5 | 1 | 0 | 0 | 0 | 1 |
| 195 | 1 | 0 | 0 | 0 | 3 |
| 200 | 1 | 0 | 0 | 1 | 0 |
| 210 | 1 | 0 | 0 | 1 | 2 |
| 220 | 1 | 0 | 0 | 2 | 0 |
| 225 | 1 | 0 | 0 | 2 | 1 |
| 240 | 0 | 1 | 1 | 0 | 0 |
| 255 | 0 | 1 | 1 | 0 | 3 |
| 260 | 0 | 1 | 1 | 1 | 0 |
| 270 | 1 | 0 | 1 | 0 | 0 |
| 280 | 1 | 0 | 1 | 0 | 0 |
| 285 | 1 | 0 | 1 | 0 | 1 |
| 300 | 0 | 0 | 3 | 0 | 0 |
| 315 | 0 | 2 | 0 | 1 | 3 |
| 320 | 1 | 1 | 0 | 0 | 0 |
| 330 | 1 | 1 | 0 | 0 | 2 |
| 340 | 0 | 1 | 2 | 0 | 0 |
| 345 | 0 | 1 | 2 | 0 | 1 |
| 360 | 2 | 0 | 0 | 0 | 0 |
| 370 | 2 | 0 | 0 | 0 | 2 |
| 380 | 2 | 0 | 0 | 1 | 0 |
| 400 | 0 | 0 | 4 | 0 | 0 |

*Assumes rounding

TABLE 5

| | Total Number of Capsules | |
|---|---|---|
| Total Daily | Current Suggested | |
| Dose | Capsule | Suggested Capsule Combinations |
| (mg)* | Combinations | of the Present Invention |
| 135 | 5 | 1 |
| 142.5 | 3 | 1 |
| 150 | 5 | 3 |
| 157.5 | 4 | 2 |
| 165 | 5 | 3 |
| 172.5 | 6 | 4 |
| 180 | 5 | 1 |
| 187.5 | 6 | 2 |
| 195 | 8 | 4 |
| 200 | 2 | 2 |
| 210 | 4 | 4 |
| 220 | 3 | 3 |
| 225 | 4 | 4 |
| 240 | 4 | 2 |
| 255 | 2 | 5 |
| 260 | 3 | 3 |
| 270 | 2 | 2 |
| 280 | 4 | 2 |
| 285 | 5 | 3 |
| 300 | 3 | 3 |
| 315 | 6 | 6 |
| 320 | 4 | 2 |
| 330 | 5 | 4 |
| 340 | 5 | 3 |
| 345 | 6 | 4 |
| 360 | 6 | 2 |
| 370 | 3 | 4 |
| 380 | 5 | 3 |
| 400 | 4 | 4 |

TABLE 6

| | Dose | | | | | | | | |
|---|---|---|---|---|---|---|---|---|---|
| | 75 mg/m² | | | 150 mg/m² | | | 200 mg/m² | | |
| BSA/Dose | Capsule | Current | 180/140 | BSA/Dose | Current | 180/140 | BSA/Dose | Current | 180/140 |
| 1.50/113 | 250 | | | 1.50/225 | | | 1.50/300 | 1 | |
| | 180 | | | | | 1 | | | |
| | 140 | | | | | | | | |
| | 100 | 1 | 1 | | 2 | | | | 3 |
| | 20 | | | | 1 | 2 | | 2 | |
| | 5 | 3 | 3 | | 1 | 1 | | 2 | |
| Total | | 4 | 4 | | 4 | 4 | | 5 | 3 |
| 1.55/116 | 250 | | | 1.55/233 | | | 1.55/310 | 1 | |
| | 180 | | | | | 1 | | | |
| | 140 | | | | | | | | |
| | 100 | 1 | 1 | | 2 | | | | 3 |
| | 20 | | | | 1 | 2 | | 3 | |
| | 5 | 3 | 3 | | 3 | 2 | | | 2 |
| Total | | 4 | 4 | | 6 | 5 | | 4 | 5 |
| 1.60/120 | 250 | | | 1.60/240 | | | 1.60/320 | 1 | |
| | 180 | | | | | | | | 1 |
| | 140 | | | | | 1 | | | 1 |
| | 100 | 1 | 1 | | 2 | 1 | | | |
| | 20 | 1 | 1 | | 2 | | | 3 | |
| | 5 | | | | | | | 2 | |
| Total | | 2 | 2 | | 4 | 2 | | 6 | 2 |
| 1.65/124 | 250 | | | 1.65/248 | 1 | | 1.65/330 | 1 | |
| | 180 | | | | | | | | 1 |
| | 140 | | | | | 1 | | | 1 |
| | 100 | 1 | 1 | | | 1 | | | |
| | 20 | 1 | 1 | | | | | 4 | |
| | 5 | 1 | 1 | | | 1 | | | 2 |
| Total | | 3 | 3 | | 1 | 3 | | 5 | 4 |
| 1.70/128 | 250 | | | 1.70/255 | 1 | | 1.70/340 | 1 | |
| | 180 | | | | | | | | |
| | 140 | | | | | 1 | | | 1 |
| | 100 | 1 | 1 | | | 1 | | | 2 |
| | 20 | 1 | 1 | | | | | 4 | |
| | 5 | 2 | 2 | | 1 | 3 | | 2 | |
| Total | | 4 | 4 | | 2 | 5 | | 7 | 3 |
| 1.75/131 | 250 | | | 1.75/263 | 1 | | 1.75/350 | 1 | |
| | 180 | | | | | | | | |
| | 140 | | | | | 1 | | | 1 |
| | 100 | 1 | 1 | | | 1 | | 1 | 2 |
| | 20 | 1 | 1 | | | 1 | | | |
| | 5 | 2 | 2 | | 3 | | | | 2 |
| Total | | 4 | 4 | | 4 | 3 | | 2 | 5 |
| 1.80/135 | 250 | | | 1.80/270 | 1 | | 1.80/360 | 1 | |
| | 180 | | | | | | | | 2 |
| | 140 | | | | | 1 | | | |
| | 100 | 1 | 1 | | | 1 | | 1 | |
| | 20 | 1 | 1 | | 1 | 1 | | | |
| | 5 | 3 | 3 | | | 2 | | 2 | |
| Total | | 5 | 5 | | 2 | 5 | | 4 | 2 |
| 1.85/139 | 250 | | | 1.85/278 | 1 | | 1.85/370 | 1 | |
| | 180 | | | | | 1 | | | 2 |
| | 140 | | 1 | | | | | | |
| | 100 | 1 | | | | 1 | | 1 | |
| | 20 | 2 | | | 1 | | | 1 | |
| | 5 | | | | 1 | | | | 2 |
| Total | | 3 | 1 | | 4 | 2 | | 3 | 4 |
| 1.90/143 | 250 | | | 1.90/285 | 1 | | 1.90/380 | 1 | |
| | 180 | | | | | 1 | | | 2 |
| | 140 | | 1 | | | | | | |
| | 100 | 1 | | | | 1 | | 1 | |
| | 20 | 2 | | | 1 | | | 1 | 1 |
| | 5 | | | | 3 | 1 | | 2 | |
| Total | | 3 | 1 | | 5 | 3 | | 5 | 3 |
| 1.95/146 | 250 | | | 1.95/293 | 1 | | 1.95/390 | 1 | |
| | 180 | | | | | 1 | | | 2 |
| | 140 | | 1 | | | | | | |
| | 100 | 1 | | | | 1 | | 1 | |
| | 20 | 2 | | | 2 | | | 2 | 1 |
| | 5 | 1 | 1 | | 1 | 3 | | | 2 |
| Total | | 4 | 2 | | 4 | 5 | | 4 | 5 |
| 2.00/150 | 250 | | | 2.00/300 | | | 2.00/400 | 1 | |
| | 180 | | | | | | | | |
| | 140 | | 1 | | | | | | |
| | 100 | 1 | | | 3 | 3 | | 1 | 4 |
| | 20 | 2 | | | | | | 2 | |
| | 5 | 2 | 2 | | | | | 2 | |
| Total | | 5 | 3 | | 3 | 3 | | 6 | 4 |
| 2.05/154 | 250 | | | 2.05/308 | | | 2.05/410 | 1 | |
| | 180 | | | | | 1 | | | |
| | 140 | | 1 | | | 1 | | | |
| | 100 | 1 | | | 3 | | | 1 | 4 |
| | 20 | 2 | | | | | | 2 | |
| | 5 | 3 | 3 | | 2 | | | 2 | |
| Total | | 6 | 4 | | 5 | 2 | | 6 | 4 |
| 2.10/158 | 250 | | | 2.10/315 | 1 | | 2.10/420 | 1 | |
| | 180 | | | | | 1 | | | 1 |
| | 140 | | 1 | | | 1 | | | 1 |
| | 100 | 1 | | | | | | 1 | 1 |
| | 20 | 3 | 1 | | 3 | | | 3 | |
| | 5 | | | | 1 | | | 2 | |
| Total | | 4 | 2 | | 5 | 2 | | 7 | 3 |
| 2.15/161 | 250 | | | 2.15/323 | 1 | | 2.15/430 | 1 | |
| | 180 | | | | | 1 | | | |
| | 140 | | 1 | | | 1 | | | 3 |
| | 100 | 1 | | | | | | 1 | |
| | 20 | 3 | 1 | | 3 | | | 4 | |
| | 5 | | | | 2 | | | | 2 |
| Total | | 4 | 2 | | 6 | 2 | | 6 | 5 |
| 2.20/165 | 250 | | | 2.20/330 | | | 2.20/440 | 1 | |
| | 180 | | | | | 1 | | | 1 |
| | 140 | | 1 | | | 1 | | | 1 |
| | 100 | 1 | | | 3 | | | 1 | 1 |
| | 20 | 3 | 1 | | 1 | | | 4 | 1 |
| | 5 | 1 | 1 | | 2 | 2 | | 2 | 2 |
| Total | | 5 | 3 | | 6 | 4 | | 8 | 4 |

### Methods of Treating Proliferative Disorders

The unit dosage forms of the present invention may be used to treat a proliferative disorder in a patient. Proliferative disorders include, but are not limited to, benign/malignant tumors such as brain tumors, prostate, lung cancer, breast cancer, ovarian, testicular cancer, liver, kidney, spleen, bladder, colorectal and/or colon cancer, head and neck, melanoma, carcinoma, sarcoma, lymphoma, mycosis fungoides as well as leukemia or other hematologic malignancies. In some embodiments, the methods are used to treat glioma, melanoma, lung cancer, lymphoma, colorectal and/or colon cancer, head and neck or ovarian cancer. In some embodiments, the methods are used to treat glioma.

In some embodiments, the unit dosage forms of the present invention are administered according to one of the dosing regimens presented in Tables 3 and 4. In some embodiments, the dosing regimen is 150-200 mg/m² per day for 5 days in a 28 day cycle. In other embodiments, the dosing regimen is 100 mg/m² per day for 14 days in a 21 day cycle. In other embodiments, the dosing regimen is 150 mg/m² per day for 7 days in a 14 day cycle.

In some embodiments, the unit dosage forms of the present invention are used to treat glioma. In some embodiments, the unit dosage forms are administered to a patient having a glioma according to one of the dosing regimens presented in Tables 3 and 4. In some embodiments, the dosing regimen is 150-200 mg/m² per day for 5 days in a 28 day cycle. In other embodiments, the dosing regimen is 100 mg/m² per day for 14 days in a 21 day cycle. In other preferred embodiments, the dosing regimen is 150 mg/m² per day for 7 days in a 14 day cycle.

In some embodiments, the unit dosage forms are administered in combination with a growth factor, Suitable growth factors include, but are not limited to, GM-CSF, G-CSF, IL-1, IL-3, IL-6, or erythropoietin. Nonlimiting growth factors include Epogen® (epoetin alfa), Procrit®. (epoetin alfa), Neupogen® (filgrastim, a human G-CSF), Aranesp® (hyperglycosylated recombinant darbepoetin alfa), Neulasta® (also branded Neupopeg, pegylated recombinant filgrastim, pegfilgrastim), Albupoietin™ (a long-acting erythropoietin), and Albugranin™ (albumin G-CSF, a long-acting G-CSF). In some embodiments, the growth factor is G-CSF.

In some embodiments, the unit dosage forms are administered in a combination therapy. In some embodiments, the unit dosage forms are administered in combination with a poly(ADP-ribose) polymerase(s) (PARP) inhibitor. The PARP inhibitor may be administered either prior to, concomitantly with or after administration of the unit dosage forms of the present invention. Suitable PARP inhibitors include but are not limited to CEP-6800 (Cephalon; described in Miknyoczki et al., Mol Cancer Ther, 2(4):371-382 (2003)); 3-aminobenzamide (also known as 3-AB; Inotek; described in Liaudet et al., Br J Pharmacol, 133(8): 1424-1430 (2001)); PJ34 (Inotek; described in Abdelkarim et al., Int J Mol Med, 7(3):255-260 (2001)); 5-iodo-6-amino-1,2-benzopyrone (also known as INH(2)BP; Inotek; described in Mabley et al., Br J Pharmacol, 133(6):909-919 (2001), GPI 15427 (described in Tentori et al., Int J Oncol, 26(2):415-422 (2005)); 1,5-dihydroxyisoquinoline (also known as DIQ; described in Walisser and Thies, Exp Cell Res, 251(2):401-413 (1999); 5-aminoisoquinolinone (also known as 5-AIQ; described in Di Paola et al., Eur J Pharmacol, 492(2-3):203-210 (2004); AG14361 (described in Bryant and Helleday, Biochem Soc Trans, 32(Pt 6):959-961 (2004); Veuger et al., Cancer Res, 63(18):6008-6015 (2003); and Veuger et al., Oncogene, 23(44):7322-7329 (2004)); ABT-472 (Abbott); INO-1001 (Inotek); AAI-028 (Novartis); KU-59436 (KuDOS; described in Farmer et al., “Targeting the DNA repair defect in BRCA mutant cells as a therapeutic strategy,” Nature, 434(7035):917-921 (2005)), and those described in Jagtap et al., Crit. Care Med, 30(5):1071-1082 (2002); Loh et al. Bioorg Med Chem Lett, 15(9):2235-2238 (2005); Ferraris et al. J Med Chem, 46(14):3138-3151 (2003); Ferraris et al., Bioorg Med Chem Lett, 13(15):2513-2518 (2003); Ferraris et al., Bioorg Med Chem, 11(17):3695-3707 (2003); Li and Zhang IDrugs, 4(7):804-812 (2001); Steinhagen et al., Bioorg Med Chem Lett, 12(21):3187-3190 (2002)); WO 02/06284 (Novantis); WO 02/06247 (Bayer); PARP inhibitors being developed by MGI Pharma Inc. (formerly Guildford Pharmaceutical, Inc.) including PARP inhibitors described in WO99/11645 (incorporated herein by reference in its entirety) including PARP inhibitors designated “1, 11b-dihydro (1)benzopyrano(4,32-de)isoquinolin-3(2H)one” such as GPI 15427 designated “10-(4-methyl-piperazin)-1-ylmethyl)-2H-7-oxa-1,2-diazabenzo[de]anthracen-3-one”; GPI 16539 designated “2-(4-methyl-piperazin-1-yl)-5H-benzo[c][1,5]napthyridin-6-one”; GPI21016, GPI 16346 and GPI 18180, GPI 6150, GPI 18078; GPI 6000 as well as 2-phenyl benzimidazole 4-carboxamides including those described by Agouron/Pfizer in WO/09524379 including such as in 16^{th} NCI-EROTC Symposium New Drugs Cancer Therapy (Amsterdam) 1998 Abs 116; Agouron 91st AACR (San Francisco) 2000, Abs. 5164 including AG-014699, AG-14361, AG-14073, as well as Kudos's KU59436, KU-0687, etc.

In some embodiments, the unit dosage forms are administered in combination with an O⁶-alkylguanine-DNA-alkyltransferase (Atase) inhibitor such as O⁶-benzylguanine (O⁶BG) or Lomeguatrib [6-(4-bromo-2-thienyl)methoxyl purin-2-amine] (a.k.a. Patrin™ or Patrin-2) described in U.S. Pat. No. 6,043,228 (Cancer Research UK) to accentuate hematopoictic toxicity. The Atase inhibitor such as O⁶BG can be administered either prior to, concomitantly with or after administration of the unit dosage forms of the present invention.

In some embodiments, the unit dosage forms are administered in combination with an anti-emetic agent. Suitable anti-emetic agents include, but are not limited to, Palonosetron, Tropisetron, Ondansetron, Granisetron, Bemesetron or a combination of at least two of the foregoing. In some embodiments, the amount of active anti-emetic substance in one dosage unit amounts to 2 to 10 mg, an amount of 5 to 8 mg active substance in one dosage unit being especially preferred. A daily dosage comprises generally an amount of active substance of 2 to 20 mg, particularly preferred is an amount of active substance of 5 to 16 mg. In some embodiments, a neurokinin-1 antagonist (NK-1 antagonist) such as aprepitant may be administered either alone or in combination with a steroid such as dexamethasone in conjunction with an anti-emetic agent. In other embodiments, the unit dosage forms are administered in combination with an NK-1 agonist alone or with a steroid. In certain embodiments, the NK-1 antagonist is one or more of the NK-1 antagonists described in U.S. Pat. No. 7,049,320 alone or in combination with one or more of an 5HT3 inhibitor and a steroid.

In some embodiments, the unit dosage forms are administered in combination with a farnesyl protein transferase inhibitor.

In other embodiments, the unit dosage forms are administered with another antineoplastic agent. Suitable antineoplastic agents include, but are not limited to, Uracil Mustard, Chlormethine, Cyclophosphamide, Ifosfamide, Melphalan, Chlorambucil, Pipobroman, Triethylenemelamine, Triethylenethiophosphoramine, Busulfan, Carmustine, Lomustine, Streptozocin, Dacarbazine, Methotrexate, 5-Fluorouracil, Floxuridine, Cytarabine, 6-Mercaptopurine, 6-Thioguanine, Fludarabine phosphate, Pentostatine, Gemcitabine, Vinblastine, Vincristine, Vindesine, Bleomycin, Dactinomycin, Daunorubicin, Doxorubicin, Epirubicin, Idarubicin, Paclitaxel, Mithramycin, Deoxycoformycin, Mitomycin-C, L-Asparaginase, Interferons, Etoposide, Teniposide 17α-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, Goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Oxaliplatin (Eloxatin®), Iressa (gefinitib, Zd1839), XELODA® (capecitabine), Tarceva® (erlotinib), Azacitidine (5-Azacytidine; 5-AzaC), and mixtures thereof.

In some embodiments, the unit dosage forms may be administered with other anti-cancer agents such as the ones disclosed in U.S. Pat. Nos. 5,824,346, 5,939,098, 5,942,247, 6,096,757, 6,251,886, 6,316,462, 6,333,333, 6,346,524, and 6,703,400, all of which are incorporated by reference.

### Methods for Assessing Methylation State of MGMT Gene

Temozolomide, as an alkylating agent, causes cell death by binding to DNA which structurally distorts the DNA helical structure preventing DNA transcription and translation. In normal cells, the damaging action of alkylating agents can be repaired by cellular DNA repair enzymes, in particular MGMT. The level of MGMT varies in tumor cells, even among tumors of the same type. The gene encoding MGMT is not commonly mutated or deleted. Rather, low levels of MGMT in tumor cells are due to an epigenetic modification; the MGMT promoter region is methylated, thus inhibiting transcription of the MGMT gene and preventing expression of MGMT.

U.S. Publication 20060100188, the entire content of which is incorporated by reference, discloses methods for treating cancer in a patient comprising administering temozolomide according to improved dosing regimen and/or schedules based on the patient's MGMT level.

In some embodiments, the dosing regimen is based upon the methylation state of the MGMT gene in a sample obtained from the patient. In some embodiments, the methylation state is assessed by a determination of whether the MGMT gene is methylated. In other embodiments, the methylation state is assessed by a quantitative determination of the level of methylation of the MGMT gene. In other embodiments, the methylation state is assessed by determination of whether MGMT protein is expressed. In yet other embodiments, the methylation states is assessed by a determination of the level of MGMT protein expressed or measurement of the enzymatic activity of MGMT in the patient sample.

Assessing whether the MGMT gene is methylated may be performed using any method known to one skilled in the art. Techniques useful for detecting methylation of a gene or nucleic acid include, but are not limited to, those described by Ahrendt et al., J. Natl. Cancer Inst., 91:332-339 (1999); Belsinky et al., Proc. Natl. Acad. Sci. U.S.A., 95:11891-11896 (1998), Clark et al. Nucleic Acids Res., 22:2990-2997 (1994); Herman et al., Proc Natl Acad Sci U.S.A., 93:9821-9826 (1996); Xiong and Laird, Nucleic Acids Res., 25:2532-2534 (1997); Eads et al., Nuc. Acids. Res., 28:e32 (2002); Cottrell et al., Nucleic Acids Res., 32:1-8 (2004).

Methylation-specific PCR (MSP) can rapidly assess the methylation status of virtually any group of CpG sites within a CpG island, independent of the use of methylation-sensitive restriction enzymes. See, MSP; Hernan et al., Proc. Natl. Acad. Sci. USA, 93(18):9821-9826 (1996); Esteller et al., Cancer Res., 59:793-797 (1999)) see also U.S. Pat. No. 5,786,146, issued Jul. 28, 1998; U.S. Pat. No. 6,017,704, issued Jan. 25, 2000; U.S. Pat. No. 6,200,756, issued Mar. 13, 2001; and U.S. Pat. No. 6,265,171, issued Jul. 24, 2001; U.S. Pat. No. 6,773,897 issued Aug. 10, 2004; the entire contents of each of which is incorporated herein by reference. The MSP assay entails initial modification of DNA by sodium bisulfite, converting all unmethylated, but not methylated, cytosines to uracil, and subsequent amplification with primers specific for methylated versus unmethylated DNA. MSP requires only small quantities of DNA, is sensitive to 0.1% methylated alleles of a given CpG island locus, and may be performed on DNA extracted from paraffin-embedded samples. MSP eliminates the false positive results inherent to previous PCR-based approaches that relied on differential restriction enzyme cleavage to distinguish methylated from unmethylated DNA. This method is very simple and can be used on small amounts of tissue or a few cells. As would be understood by those skilled in the art, if the gene encoding MGMT is not methylated, the MGMT protein is expressed and can be detected (e.g., by Western blot, immuno-histochemical techniques or enzymatic assays for MGMT activity, etc.) as detailed below herein.

An illustrative example of a Western blot assay useful for this embodiment of the invention to measure the level of MGMT protein in patient samples is presented in U.S. Pat. No. 5,817,514 by Li et al., the entire disclosure of which is incorporated herein by reference. Li et al. described monoclonal antibodies able to specifically bind either to native human MGMT protein or to human MGMT protein having an active site which is alkylated.

An illustrative example of an immunohistochemical technique useful for this embodiment of the invention to measure the level of MGMT protein in patient samples is presented in U.S. Pat. No. 5,407,804, the entire disclosure of which is incorporated herein by reference. Monoclonal antibodies are disclosed which are able to specifically bind to the MGMT protein in single cell preparations (immunohistochemical staining assays) and in cell-extracts (immunoassays). The use of fluorescent read out coupled with digitization of the cell image is described and allows for quantitative measurement of MGMT levels in patient and control samples, including but not limited to tumor biopsy samples.

Useful techniques for measuring the enzymatic activity of MGMT protein include but are not limited to methods described by: Myrnes et al., Carcinogenesis, 5:1061-1064 (1984); Futscher et al., Cancer Comm., 1: 65-73 (1989); Kreklaw et al., J. Pharmacol. Exper. Ther., 297(2):524-530 (2001); and Nagel et al., Anal. Biochem., 321(1):38-43 (2003), the entire disclosures of which are incorporated herein in their entireties.

The level of MGMT protein expressed in a sample obtained from a patient may be assessed by measuring MGMT protein, e.g., by Western blot using an antibody specific to MGMT, see for example, U.S. Pat. No. 5,817,514. The level of MGMT expressed in a sample may also be assessed by measuring the MGMT protein using an immunohistochemistry technique on a defined number of patient cells, e.g., employing a labeled antibody specific for MGMT and comparing the level with that expressed by the same defined number of normal lymphocytes known to express MGMT (see, for example, U.S. Pat. No. 5,407,804 by Yarosh for a description of useful quantitative immunohistochemical assays). Alternatively, the level of MGMT may be assessed by enzymatic assay, i.e., the ability to methylate the O⁶ or N⁷ guanine position of DNA. In each of these methods, the measured level of MGMT protein expressed is compared to that expressed by normal lymphocytes known to express MGMT. Patient MGMT protein levels are classified as follows. Low=0-30% of the MGMT expressed by normal lymphocytes; Moderate=31-70% of the MGMT expressed by normal lymphocytes; and High=71-300% or higher of the MGMT expressed by normal lymphocytes. Patient MGMT levels are classified as follows: Low=0-30% of the MGMT enzymatic activity of normal lymphocytes; Moderate=31-70% of the MGMT enzymatic activity of normal lymphocytes; and High=71-300% or higher of the MGMT enzymatic activity of normal lymphocytes.

The specific activity of MGMT may be assessed and based on a comparison with cell lines known to express MGMT classified as follows: Low=less than 20 fmol/mg; Moderate=20-60 fmol/mg; or High=greater than 60 fmol/mg; where the specific activity of MGMT in LOX cells is 6-9 fmol/mg, in DAOY cells is 60-100 fmol/mg, and in A375 cells is 80-150 fmol/mg.

The level of methylation of MGMT may be assessed by quantitative determination of the methylation of the gene encoding MGMT. The quantitative technique called COBRA (Xiong et al., Nuc. Acids Res., 25:2532-2534 (1997)) may be used in this determination. The “methyl light” technique of Eads et al., Nuc. Acids Res., 28(8):e32 (2000); U.S. Pat. No. 6,331,393 may also be used. The level of methylation of gene encoding MGMT in cells of the patient is compared to that of an equivalent number of cells of normal lymphocytes known to express MGMT. As would be understood by those skilled in the art, normal lymphocytes expressing MGMT have a low level of methylation of the MGMT gene; conversely, cells with high levels of methylation of the MGMT gene express low levels of the MGMT protein (see for example, Costello et al., J. Biol. Chem., 269(25):17228-17237 (1994); Qian et al., Carcinogen, 16(6):1385-1390 (1995)). Patient methylated MGMT gene levels are classified as follows. Low=0-20% of the CpGs in the promoter region of the MGMT gene are methylated; Moderate=21-50% of the CpGs in the promoter region of the MGMT gene are methylated; and High=1-100% of the CpGs in the promoter region of the MGMT gene are methylated.

COBRA may be used to determine quantitatively DNA methylation levels at specific gene loci in small amounts of genomic DNA. Restriction enzyme digestion is used to reveal methylation-dependent sequence differences in PCR products of sodium bisulfite-treated DNA. (Tano et al., Proc. Natl. Acad. Sci. USA, 87:686-690 (1990) describe isolation and sequence of the human MGMT gene). Methylation levels in original DNA sample are represented by the relative amounts of digested and undigested PCR product in a linearly quantitative fashion across a wide spectrum of DNA methylation levels. This technique may be reliably applied to DNA obtained from microdissected paraffin-embedded tissue samples. COBRA thus combines the powerful features of ease of use, quantitative accuracy, and compatibility with paraffin sections.

An illustrative example of a RT-PCR assay useful for assessing the level of MGMT mRNA is described in Watts et al., Mol. Cell. Biol., 17(9):5612-5619 (1997). In brief, total cellular RNA is isolated by guanidium isothiocyanate cell lysis followed by centrifugation through a 5.7 M CsCl gradient for 2.5 hr at 205,000×g. RNA is quantitated in a Beckman TL-100 spectrophotometer by measurements of absorbance at 260 nm. Total cellular RNA is reverse transcribed by incubating a 40 μl reaction mixture composed of 200 ng of RNA; 1×PCR buffer (10 mM Tris [pH 8.3], 50 mM KCl, 1.5 mM MgCl₂); 1 mM each dATP, dCTP, dGTP, and dTTP; 200 pmol of random hexamer, 40 U of RNasin, and 24 U of avian myeloblastosis virus reverse transcriptase (Boehringer Mannheim, Indianapolis, Ind.) at 42° C. for 60 min. The reaction is then stopped by incubation at 99° C. for 10 min. MGMT-specific PCR is performed by adding 80 μl of amplification reaction buffer (1×PCR buffer, 25 pmol of MGMT-specific primers and/or a control sequence, and 2 U of Taq DNA polymerase) to 20 μl of the reverse transcription reaction mixture followed by incubation at 94° C. for 5 min; 30 cycles of 94° C. for 1 min, 60° C. for 15 s, and 72° C. for 1 min; a final extension at 72° C. for 5 min; and a quick chill to 4° C. For example, the upstream primer sequence from exon 4 (nt 665 to 684) of the MGMT gene can be used. Nucleotide positions can be derived from the cDNA sequence (Tano et al., Proc. Natl. Acad. Sci. USA, 87:686-690 (1990)). A control primer sequence can be employed in the same cDNA reaction (e.g., primers for the histone 3.3 gene). For analysis, 10% of the respective PCR products are separated through a 3% agarose gel and visualized by ethidium bromide staining.

Methods of Treating a Patient with a Glioma Based Upon Methylation State of MGMT Gene

The unit dosage forms of the present invention are used to treat a patient having a glioma. In some embodiments, the dosing regimen is based upon the detection of methylated MGMT gene in a sample. When methylation of the MGMT gene is detected in a sample obtained from a patient having a glioma, the dosing regimen is 150-200 mg/m² per day for 5 days in a 28 day cycle. When methylation is not detected, the dosing regimen is (i) 100 mg/m² per day for 14 days in a 21 day cycle; (ii) 150 mg/m² per day for 7 days in a 14 day cycle; or (iii) 100 mg/m² per day for 21 days in a 28 day cycle. In some embodiments, the sample is a tumor biopsy sample. In some embodiments, the MGMT gene is detected using MSP.

In other embodiments, the dosing regimen is based upon the presence or absence of MGMT protein. In those embodiments basedupon the presence or absence of the MGMT protein, when the MGMT protein is not detected in a sample obtained from a patient having a glioma, the dosing regimen is 150-200 mg/m² per day for 5 days in a 28 day cycle; when the MGMT protein is detected, the dosing regimen is selected from (i) 100 mg/m² per day for 14 days in a 21 day cycle; 150 mg/m² per day for 7 days in a 14 day cycle; or (iii) 100 mg/m² per day for 21 days in a 28 day cycle. In some embodiments, the sample is a tumor biopsy sample. In some embodiments, the MGMT protein is detected using Western blot immunoassay, an immunohistochemical technique, or an enzymatic assay for MGMT protein.

In yet other embodiments, the dosing regimen is based upon the level or activity of the MGMT protein detected in a sample. When the level or enzymatic activity of the MGMT protein detected in a sample obtained from the patient is Low, compared to that of normal lymphocytes, the dosing regimen is (i) 150-200 mg/m² per day for 5 days in a 28 day cycle; or (ii) 250 mg/m² per day for 5 days in a 28 day cycle in combination with a growth factor. When the level or enzymatic activity of the MGMT protein is Moderate, the dosing regimen is (i) 100 mg/m² per day for 14 days in a 28 day cycle; (ii) 300 mg/m² per day for 5 days in a 28 day cycle in combination with a growth factor; (iii) 75 mg/m² per day for 21 days in a 28 day cycle; or (iv) 75 mg/m² per 42 days in a 56 day cycle. When the level or enzymatic activity of the MGMT protein detected in a sample obtained from the patient is High, the dosing regimen is selected from (i) 100 mg/m² per day for 14 days in a 21 day cycle; (ii) 150 mg/m² per day for 7 days in a 14 day cycle; or (iii) 100 mg/m² per day for 21 days in a 28 day cycle. In some embodiments, the sample is a tumor biopsy sample.

Methods of Treating a Patient with Certain Proliferative Disorders Based Upon Methylation State of MGMT Gene

In some embodiments, the unit dosage forms are used to treat patients having a proliferative disorder selected from melanoma, lung cancer, lymphoma, head cancer, neck cancer, ovarian cancer, colorectal cancer, colon cancer and esophogeal cancer. In some embodiments, the dosing regimen is based upon the detection of methylated MGMT gene in a sample. When methylation of the MGMT gene is not detected in a sample obtained from a patient, the dosing regimen is (i) 100 mg/m per day for 14 days in a 21 day cycle; (ii) 150 mg/m² per day for 7 days in a 14 day cycle; or (iii) 100 mg/m² per day for 21 days in a 28 day cycle. In some embodiments, the sample is a tumor biopsy sample. In some embodiments, the MGMT gene is detected using MSP.

In other embodiments, the dosing regimen is based upon the detection of MGMT protein in a sample. When the MGMT protein is not detected, the dosing regimen is selected from (i) 100 mg/m² per day for 14 days in a 21 day cycle; 150 mg/m² per day for 7 days in a 14 day cycle; or (iii) 100 mg/m² per day for 21 days in a 28 day cycle. In some embodiments, the sample is a tumor biopsy sample. In some embodiments, the MGMT protein is detected using Western blot immunoassay, an immunohistochemical technique, or an enzymatic assay for MGMT protein.

In yet other embodiments, the dosing regimen is based upon the level or activity of the MGMT protein detected in a sample. When the level or enzymatic activity of the MGMT protein detected in a sample obtained from the patient is Low or Moderate, compared to that of normal lymphocytes, the dosing regimen is (i) 100 mg/m² per day for 14 days in a 21 day cycle (ii) 150 mg/m² per day for 7 days in a 14 day cycle; or (iii) 100 mg/m² per day for 21 days in a 28 day cycle. In some embodiments, the sample is a tumor biopsy sample.

### Kits Comprising the Unit Dosage Forms of the Present Invention

The unit dosage forms of the present invention may be included in a kit. The kit may be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or metal or plastic foil, such as a blister pack with individual doses for pressing out of the pack or a sachet or envelope-like container that may be ripped open to release the unit dosage form according to a therapeutic schedule. In some embodiments, the kit is a blister pack. In other embodiments, the kit may include more than one container to market a single dosage form. For example, capsules or tablets may be contained in a bottle, which is in turn contained within a box. In some embodiments, the kit is air-tight. In some embodiments, the kit is made from a light resistant material.

In some embodiments, the kit comprise one or more unit dosage forms comprising about 5, 20, 100 140 or 180 mg of temozolomide. In some embodiments, the kit is a blister pack. The different dosage forms may be arranged in the form of capsules, tablets or pills in rows lying next to one another in the blister pack. At the time indicated by the physician, the patient would in each case successively take a capsule, tablet or pill from each row within a short length of time (in particular within 5 minutes).

In some embodiments, the kit may be an DA approved kit. In some embodiments, the kit is accompanied by instructions for administration. The kit may also be accompanied by a notice associated with the container in a form prescribed by a governmental agency regulating the manufacture, use or sale of pharmaceuticals, which notice is reflective of approval by the agency of the form of the compositions or of human or veterinary administration. Such notice, for example, may be of the labeling approved by the U.S. Food and Drug Administration for prescription drugs or of an approved product insert. The unit dosage forms may also be prepared, placed in an appropriate container, and labeled for treatment of an indicated condition.

In order that this invention be more fully understood, the following example are set forth. This example is for the purpose of illustration only and is not to be construed as limiting the scope of the invention in any way.

### EXAMPLE

Patients with glioma were enrolled in a clinical trial to receive treatment with temozolomide. The drug was administered at a dosage of 75 mg/m², 150 mg/m² or 200 mg/m² using only the currently available suggested capsule combinations of Table 6 ie. 5, 20 and 100 mg capsules. The daily dosage was determined by the patient's BSA. The majority of the patients enrolled in the trial had BSAs ranging from 1.8 to 2.0 (149 out of 284). Upon reviewing the data regarding the pill burden, the present inventors unexpectedly discovered that adding unit dosages of about 140 and/or 180 mg of temozolomide would be beneficial to reduce patient pill burden. See Table 7.

As shown in Table 7, the pill burden would be decreased for patients receiving the unit dosage forms of the present invention in 16 of the 24 possible dosing regimens. The decreased pill burden would be especially significant in patients having a BSA between 1.8-2.0. For these patients, the pill burden would be decreased in 6 of the possible 8 regimens.

TABLE 7

| | | Number of Dosing | | |
|---|---|---|---|---|
| | | Regimens | | |
| BSA | Number of Patients | Lower | Same | Higher |
| 1.4 | 6 | — | — | — |
| 1.5 | 9 | 1 | 2 | 0 |
| 1.6 | 23 | 2 | 1 | 0 |
| 1.7 | 40 | 1 | 1 | 1 |
| 1.8 | 40 | 1 | 1 | 1 |
| 1.9 | 66 | 3 | 0 | 0 |
| 2.0 | 43 | 2 | 1 | 0 |
| 2.1 | 26 | 3 | 0 | 0 |
| 2.2 | 21 | 3 | 0 | 0 |
| 2.3 | 5 | — | — | — |
| 2.4 | 4 | — | — | — |
| 2.5 | 1 | — | — | — |

What is claimed is:

1. A unit dosage form of temozolomide, comprising about 140 mg of temozolomide and a pharmaceutically acceptable carrier.

2. A unit dosage form of temozolomide, comprising about 180 mg of temozolomide and a pharmaceutically acceptable carrier.

3. The unit dosage form according to claim 1 or 2, wherein the formulation is a capsule.

4. The unit dosage form according to claim 3, wherein the capsule is color-coded.

5. The unit dosage form according to claim 4, wherein the 140 mg temozolomide capsule differs in color from the 180 mg temozolomide capsule.

6. A method of increasing patient compliance with a regimen, comprising the step of administering to the patient the unit dosage form according to claim 1 or 2 or a combination thereof.

7. A method for treating a patient having a glioma, comprising the step of administering to the patient the unit dosage form according to claim 1 or 2 or a combination thereof.

8. A method for treating a patient having a proliferative disorder selected from at lease one of the group consisting of melanoma, lung cancer, lymphoma, head cancer, neck cancer, ovarian cancer, colorectal cancer, colon cancer and esophageal cancer comprising the step of administering to the patient the unit dosage form according to claim 1 or 2 or a combination thereof.

9. A method for treating a patient having a glioma, comprising, the step of administering to the patient the unit dosage form according to claim 1 or 2 or a combination thereof in a regimen selected from the group consisting of,
(a) 150-200 mg/m² per day for 5 days in a 28 day cycle;
(b) 250 mg/m² per day for 5 days in a 28 day cycle in combination with a growth factor;
(c) 100 mg/m² per day for 14 days in a 28 day cycle;
(d) 300 mg/m² per day for 5 days in a 28 day cycle in combination with a growth factor;
(e) 75 mg/m² per day for 21 days in a 28 day cycle;
(f) 75 mg/m² per 42 days in a 56 day cycle;
(g) 85 mg/m² per 21 days in a 28 day cycle;
(h) 350 mg/m² per day for 5 days in a 28 day cycle in combination with a growth factor;
(i) 100 mg/m² per day for 14 days in a 21 day cycle;
(j) 400 mg/m² per day for 5 days in a 28 day cycle in combination with a growth factor;
(k) 150 mg/m² per day for 7 days in a 14 day cycle;
(l) 100 mg/m² per day for 21 days in a 28 day cycle;
(m) 150 mg/m² per day for 14 days in a 28 day cycle;
(n) 75 mg/m² per day daily;
(o) 450 mg/m² per day for 5 days in a 28 day cycle in combination with a growth factor;
(p) 150 mg/m² per day for 14 days in a 21 day cycle;
(q) 100 mg/m² per day daily;
(r) 250 mg/m² per day for 7 days in a 14 day cycle in combination with a growth factor; and
(s) 300 mg/m² per day for 7 days in a 14 day cycle in combination with a growth factor.

10. The method according to claim 9, wherein the regimen is selected from the group consisting of:
(a) 150-200 mg/m² per day for 5 days in a 28 day cycle;
(b) 100 mg/m² per day for 14 days in a 21 day cycle; and
(c) 150 mg/m² per day for 7 days in a 14 day cycle.

11. A method for treating a patient having a glioma, comprising the step of administering to the patient the unit dosage form according to claim 1 or 2 or a combination thereof in a regimen selected from the group consisting of:
(a) when methylation of the MGMT gene is detected in a sample obtained from the patient:
(i) 150-200 mg/m² per day for 5 days in a 28 day cycle; and
(b) when methylation of the MGMT gene is not detected in a sample obtained from the patient:
(i) 100 mg/m² per day for 14 days in a 21 day cycle;
(ii) 150 mg/m² per day for 7 days in a 14 day cycle; or
(iii) 100 mg/m² per day for 21 days in a 28 day cycle.

12. The method according to claim 11, wherein the sample is a tumor biopsy sample.

13. The method according to claim 11 wherein the MGMT gene is detected using methylation-specific PCR.

14. A method for treating a patient having a glioma, comprising the step of administering to the patient the unit dosage form according to claim 1 or 2 or a combination thereof in a regimen selected from the group consisting of:
(a) when the MGMT protein is not detected in a sample obtained from the patient:
(i) 150-200 mg/m² per day for 5 days in a 28 day cycle; and
(b) when the MGMT protein is detected in a sample obtained from the patient:
(i) 100 mg/m² per day for 14 days in a 21 day cycle;
(ii) 150 mg/m² per day for 7 days in a 14 day cycle; or
(iii) 100 mg/m² per day for 21 days in a 28 day cycle.

15. The method according to claim 14, wherein the sample is a tumor biopsy sample.

16. The method according to claim 14, wherein the MGMT protein is detected using Western blot immunoassay, an immunohistochemical technique, or an enzymatic assay for MGMT protein.

17. A method for treating a patient having a glioma, comprising the step of administering to the patient the unit dosage form according to claim 1 or 2 or a combination thereof in a regimen selected from the group consisting of:
(a) when the level or enzymatic activity of the MGMT protein detected in a sample obtained from the patient is Low, compared to that of normal lymphocytes:
(i) 150-200 mg/m² per day for 5 days in a 28 day cycle; or
(ii) 250 mg/m² per day for 5 days in a 28 day cycle in combination with a growth factor;
(b) when the level or enzymatic activity of the MGMT protein detected in a sample obtained from the patient is Moderate, compared to that of normal lymphocytes:
(i) 100 mg/m² per day for 14 days in a 28 day cycle;
(ii) 300 mg/m² per day for 5 days in a 28 day cycle in combination with a growth factor;
(iii) 75 mg/m² per day for 21 days in a 28 day cycle; or
(iv) 75 mg/m² per 42 days in a 56 day cycle; and
(c) when the level or enzymatic activity of the MGMT protein detected in a sample obtained from the patient is High, compared to that of normal lymphocytes:
(i) 100 mg/m² per day for 14 days in a 21 day cycle;
(ii) 150 mg/m² per day for 7 days in a 14 day cycle; or
(ii) 100 mg/m² per day for 21 days in a 28 day cycle.

18. The method according to claim 17, wherein the sample is a tumor biopsy sample.

19. A method for treating a patient having a melanoma, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when methylation of the MGMT gene is detected in a sample obtained from the patient.

20. The method according to claim 19, wherein the sample is a tumor biopsy sample.

21. The method according to claim 19, wherein the MGMT gene is detected using methylation-specific PCR.

22. A method for treating a patient having a melanoma, comprising the step of administering to the patient the unit dosage form according to claim 1 or 2 or a combination thereof in a regimen selected from the group consisting of:
(a) 100 mg/m² per day for 14 days in a 21 day cycle,
(b) 150 mg/m² per day for 7 days in a 14 day cycle; and
(c) 100 mg/m² per day for 21 days in a 28 day cycle;
when the MGMT protein is not detected in a sample obtained from the patient.

23. The method according to claim 22, wherein the sample is a tumor biopsy sample.

24. The method according to claim 22, wherein the MGMT protein is detected using Western blot immunoassay, an immunohistochemical technique, or an enzymatic assay for MGMT protein.

25. A method for treating a patient having a melanoma, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when the level or enzymatic activity of the MGMT protein detected in a sample obtained from the patient is Low or Moderate, compared to that of normal lymphocytes.

26. The method according to claim 25, wherein the sample is a tumor biopsy sample.

27. A method for treating a patient having a lung cancer, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when methylation of the MGMT gene is detected in a sample obtained from the patient.

28. The method according to claim 27, wherein the sample is a tumor biopsy sample.

29. The method according to claim 27, wherein the MGMT gene is detected using methylation-specific PCR.

30. A method for treating a patient having a lung cancer, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when the MGMT protein is not detected in a sample obtained from the patient.

31. The method according to claim 30, wherein the sample is a tumor biopsy sample.

32. The method according to claim 30, wherein the MGMT protein is detected using Western blot immunoassay, an immunohistochemical technique, or an enzymatic assay for MGMT protein.

33. A method for treating a patient having a lung cancer, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when the level or enzymatic activity of the MGMT protein detected in a sample obtained from the patient is Low or Moderate, compared to that of normal lymphocytes.

34. The method according to claim 33, wherein the sample is a tumor biopsy sample.

35. A method for treating a patient having a lymphoma, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when methylation of the MGMT gene is detected in a sample obtained from the patient.

36. The method according to claim 35, wherein the sample is a tumor biopsy sample.

37. The method according to claim 35, wherein the MGMT gene is detected using methylation-specific PCR.

38. A method for treating a patient having a lymphoma, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when the MGMT protein is not detected in a sample obtained from the patient.

39. The method according to claim 38, wherein the sample is a tumor biopsy sample.

40. The method according to claim 38, wherein the MGMT protein is detected using Western blot immunoassay, an immunohistochemical technique, or an enzymatic assay for MGMT protein.

41. A method for treating a patient having a lymphoma, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when the level or enzymatic activity of the MGMT protein detected in a sample obtained from the patient is Low or Moderate, compared to that of normal lymphocytes.

42. The method according to claim 41, wherein the sample is a tumor biopsy sample.

43. A method for treating a patient having head and neck cancer, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when methylation of the MGMT gene is detected in a sample obtained from the patient.

44. The method according to claim 43, wherein the sample is a tumor biopsy sample.

45. The method according to claim 43, wherein the MGMT gene is detected using methylation-specific PCR.

46. A method for treating a patient having head and neck cancer, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when the MGMT protein is not detected in a sample obtained from the patient.

47. The method according to claim 46, wherein the sample is a tumor biopsy sample.

48. The method according to claim 46, wherein the MGMT protein is detected using Western blot immunoassay, an immunohistochemical technique, or an enzymatic assay for MGMT protein.

49. A method for treating a patient having head and neck cancer, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when the level or enzymatic activity of the MGMT protein detected in a sample obtained from the patient is Low or Moderate, compared to that of normal lymphocytes.

50. The method according to claim 49, wherein the sample is a tumor biopsy sample.

51. A method for treating a patient having ovarian cancer, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when methylation of the MGMT gene is detected in a sample obtained from the patient.

52. The method according to claim 51, wherein the sample is a tumor biopsy sample.

53. The method according to claim 51, wherein the MGMT gene is detected using methylation-specific PCR.

54. A method for treating a patient having ovarian cancer, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when the MGMT protein is not detected in a sample obtained from the patient.

55. The method according to claim 54, wherein the sample is a tumor biopsy sample.

56. The method according to claim 54, wherein the MGMT protein is detected using Western blot immunoassay, an immunohistochemical technique, or an enzymatic assay for MGMT protein.

57. A method for treating a patient having ovarian cancer, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when the level or enzymatic activity of the MGMT protein detected in a sample obtained from the patient is Low or Moderate, compared to that of normal lymphocytes.

58. The method according to claim 57, wherein the sample is a tumor biopsy sample.

59. A method for treating a patient having colorectal and/or colon cancer, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when methylation of the MGMT gene is detected in a sample obtained from the patient.

60. The method according to claim 59, wherein the sample is a tumor biopsy sample.

61. The method according to claim 59, wherein the MGMT gene is detected using methylation-specific PCR.

62. A method for treating a patient having colorectal and/or colon cancer, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when the MGMT protein is not detected in a sample obtained from the patient.

63. The method according to claim 62, wherein the sample is a tumor biopsy sample.

64. The method according to claim 62, wherein the MGMT protein is detected using Western blot immunoassay, an immunohistochemical technique, or an enzymatic assay for MGMT protein.

65. A method for treating a patient having colorectal and/or colon cancer, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when the level or enzymatic activity of the MGMT protein detected in a sample obtained from the patient is Low or Moderate, compared to that of normal lymphocytes.

66. The method according to claim 65, wherein the sample is a tumor biopsy sample.

67. A method for treating a patient having esophageal cancer, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when methylation of the MGMT gene is detected in a sample obtained from the patient.

68. The method according to claim 67, wherein the sample is a tumor biopsy sample.

69. The method according to claim 67, wherein the MGMT gene is detected using methylation-specific PCR.

70. A method for treating a patient having esophageal cancer, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when the MGMT protein is not detected in a sample obtained from the patient.

71. The method according to claim 70, wherein the sample is a tumor biopsy sample.

72. The method according to claim 70, wherein the MGMT protein is detected using Western blot immunoassay, an immunohistochemical technique, or an enzymatic assay for MGMT protein.

73. A method for treating, a patient having esophageal cancer, comprising the step of administering to the patient the unit dosage form according to claim 1 or 2 or a combination thereof in a regimen selected from the group consisting of:
(a) 100 mg/m² per day for 14 days in a 21 day cycle;
(b) 150 mg/m² per day for 7 days in a 14 day cycle; and
(c) 100 mg/m² per day for 21 days in a 28 day cycle;
when the level or enzymatic activity of the MGMT protein detected in a sample obtained from the patient is Low or Moderate, compared to that of normal lymphocytes.

74. The method according to claim 73, wherein the sample is a tumor biopsy sample.

75. A kit comprising the unit dosage for of claim 1.

76. The kit according to claim 74, further comprising the unit dosage for of claim 2.

77. A kit comprising the unit dosage form of claim 2.

78. The kit according to any one of claims 75-77, wherein the kit further comprising one or more unit dosage forms comprising about 5, 20 or 100 mg of temozolomide.

79. The kit according to any one of claims 75-77, wherein the kit is a blister pack.

80. The kit according to claim 78, wherein the kit is a blister pack.

81. The kit according to claim 79, wherein the unit dosage form is a capsule.

82. The kit according to claim 80, wherein the unit dosage form is a capsule
